(19) United States
(12) Patent Application Publication
(10) Pub. No.: US 2024/0226350 A1
(43) Pub. Date: Jul. 11, 2024

(54) UV Integrated Disinfecting Cap

(71) Applicant: Bard Access Systems, Inc., Salt Lake City, UT (US)

(72) Inventors: Dustin Payne, West Jordan, UT (US); Timothy M. Snelling, Salt Lake City, UT (US); Matthew J. Prince, Herriman, UT (US); Craig Hayden, Sandy, UT (US)

(21) Appl. No.: 18/094,760

(22) Filed: Jan. 9, 2023

(51) Int. Cl. A61L 2/10; A61L 2/24

(52) U.S. Cl. CPC A61L 2/10; A61L 2/24

(57) ABSTRACT

A disinfection device for disinfecting a hub of a medical device includes a housing having a first end and a second end, a power source, an ultraviolet light source, and a connection mechanism located at the first end of the housing, wherein connection of the hub of the medical device to the disinfection device initiates a disinfection process configured to disinfect the hub. the ultraviolet source may be a light emitting diode with a peak of wavelength within the range of 100-400 nm (specific implementations being 225-264 nm) and the connection mechanism includes one or more of a screw thread, a channel, ridge, a friction fit connector, a magnet, or a ferromagnetic material. The disinfection device may include a port located on the second end, wherein the one or more port is configured for coupling to an external device and receiving a charge or power supply from the external device.

### BACKGROUND

One challenge of modern medical treatment is control of infection and the spread of microbial organisms. One area where this challenge is constantly presented is in infusion therapy procedures. Infusion therapy is one of the most common health care procedures. Hospitalized, home care, and other patients receive fluids, pharmaceuticals, and blood products via a vascular access device inserted into the vascular system. Infusion therapy may be used to treat an infection, provide anesthesia or analgesia, provide nutritional support, treat cancerous growths, and maintain blood pressure and heart rhythm, or many other clinically significant uses.

In some instances, an implanted port may be placed under a patient's skin to enable intravenous (IV) treatments and transfusions directly into a vein. As convenient as implanted ports are, the spread of microbial organisms into the patient's vein through the implanted port is of great concern. Similar concerns exist with connectors of other medical apparatuses as well such as connectors at proximal or distal ends of a catheter. Thus, what is needed is a method, system, and apparatus for disinfecting medical apparatuses thereby preventing the spread of microbial organisms into the patient body.

### SUMMARY

Briefly summarized, embodiments of the present invention are directed to a disinfection device for disinfecting a hub of a medical device. The disinfection device may include a housing having a first end and a second end, a power source, an ultraviolet source, and a connection mechanism located at the first end of the housing. In some aspects, the ultraviolet source may be a light emitting diode with a peak of wavelength within the range of 100-400 nm. In some embodiments, the ultraviolet light source may be a germicidal ultraviolet light (UV-C) having a wavelength range of substantially 225-264 nm. In some aspects, the connection mechanism may include one or more screw thread, channel, ridge, friction fit, magnet, and ferromagnetic material. In some aspects, the device may include one or more port located on the second end.

In some aspects, the one or more port may be configured for coupling to an external device. In some aspects, the external device may be capable of charging the power source. In some aspects, the power source may include one or more battery, capacitor, and induction coil. In some aspects, the connection mechanism may be configured for attaching the device to a hub of a medical device. In some aspects, the device may include an adapter.

In some aspects, the connection mechanism may be configured for attaching to the adapter, and the adapter may be configured for attaching to a hub of a medical device. In some aspects, the device may include at least one indicator. In some aspects, the at least one indicator may reflect a power level of the power source. In some aspects, the at least one indicator may be at least one visible light spectrum light emitting diode. In some aspects, the at least one indicator may reflect a status of the device.

In some aspects, the status may be indicative of one or more power setting, timing, and intensity. In some aspects, connection of a hub of a medical device with the connection mechanism may initiate disinfection of the hub. In some aspects, the device may include a transmitter or transceiver. In some aspects, the transmitter or transceiver may include a radio-frequency identification (RFID) or BLUETOOTH® enabled transmitter or transceiver.

These and other features of embodiments of the present invention will become more fully apparent from the following description and appended claims or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is a perspective view of a device for disinfecting a hub in accordance with an embodiment;

FIG. 2 is a front view of the device of FIG. 1;

FIG. 3 is an end view of the device of FIG. 1;

FIG. 4 is a perspective view of the device for disinfecting a hub of FIG. 1 connected to a male hub member;

FIG. 5 is a perspective view of the device for disinfecting a hub of FIG. 1 connected to a female hub member;

FIG. 6A is a perspective view of an adapter for use with a device for disinfecting a hub in accordance with an embodiment;

FIG. 6B is a perspective view of an adapter for use with a device for disinfecting a hub in accordance with an embodiment;

FIG. 6C is a perspective view of an adapter for use with a device for disinfecting a hub in accordance with an embodiment;

FIG. 7 is a perspective view of a device for disinfecting a hub including an adapter connected to a male hub member; and

FIG. 8 is a perspective view of a device for disinfecting a hub including an adapter connected to a female hub member.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a widget disclosed herein includes a portion of the widget intended to be near a user (e.g., a holder of the widget). Likewise, a “proximal length” of, for example, the widget includes a length of the widget intended to be near the user. A “proximal end” of, for example, the widget includes an end of the widget intended to be near the user. The proximal portion, the proximal end portion, or the proximal length of the widget can include the proximal end of the widget; however, the proximal portion, the proximal end portion, or the proximal length of the widget need not include the proximal end of the widget. That is, unless context suggests otherwise, the proximal portion, the proximal end portion, or the proximal length of the widget is not a terminal portion or terminal length of the widget.

With respect to “distal,” a “distal portion” or a “distal end portion” of, for example, a widget disclosed herein includes a portion of the widget intended to be opposite the user with respect to the proximal portion (e.g., “away” from the user). Likewise, a “distal length” of, for example, the widget includes a length of the widget intended to be opposite the proximal portion and away from the user. A “distal end” of, for example, the widget includes an end of the widget intended to be opposite the proximal end. The distal portion, the distal end portion, or the distal length of the widget can include the distal end of the widget; however, the distal portion, the distal end portion, or the distal length of the widget need not include the distal end of the widget. That is, unless context suggests otherwise, the distal portion, the distal end portion, or the distal length of the widget is not a terminal portion or terminal length of the widget.

The term “logic” may be representative of hardware, firmware or software that is configured to perform one or more functions. As hardware, the term logic may refer to or include circuitry having data processing and/or storage functionality. Examples of such circuitry may include, but are not limited or restricted to, a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical, or other form of propagated signals such as carrier waves, infrared signals, or digital signals). Examples of a non-transitory storage medium may include, but are not limited or restricted to, a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Embodiments described herein are generally directed to a disinfection device capable of disinfecting the hubs of medical devices. For example, the disinfection device may be used to disinfect one or more hub of a multi-lumen catheter, infusion catheter, or other medical device. In some aspects, the one or more hubs may include a male or female Luer connector.

The germicidal or biocidal effects of ultraviolet (UV) radiation have been known since the late 19th century. In recent years the use of UV radiation has gained broad acceptance in the fields of water and air purification and has found some limited use in food processing and medical device sterilization.

UV light consists of high energy photons which occupy the 200 to 400 nanometer wavelengths of the electromagnetic spectrum. This means that UV light emits slightly less energy than soft X-ray radiation, but significantly more than visible light. UV energy does not directly kill pathogens, but rather causes a photochemical reaction with the genetic structure which inhibits the ability of the pathogens to reproduce, therefore, in effect, killing the pathogen.

The amount of energy delivered by UV light is inversely proportional to its wavelength, therefore, the shorter the wavelength, the greater the energy produced. In general, the UV light portion of the spectrum is made up of three segments: UV-A (315-400 nm), used for sun-tanning lamps; UV-B (280-315 nm); and UV-C (200-280 nm). The UV-B and UV-C regions contain wavelengths with the best biocidal action. Studies have shown that the wavelengths most effective in killing microbes are between 250-265 nm.

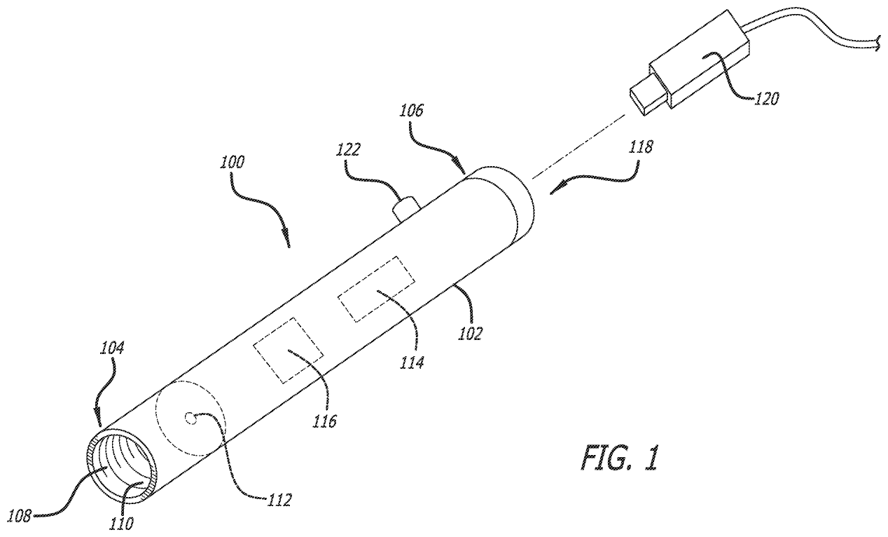

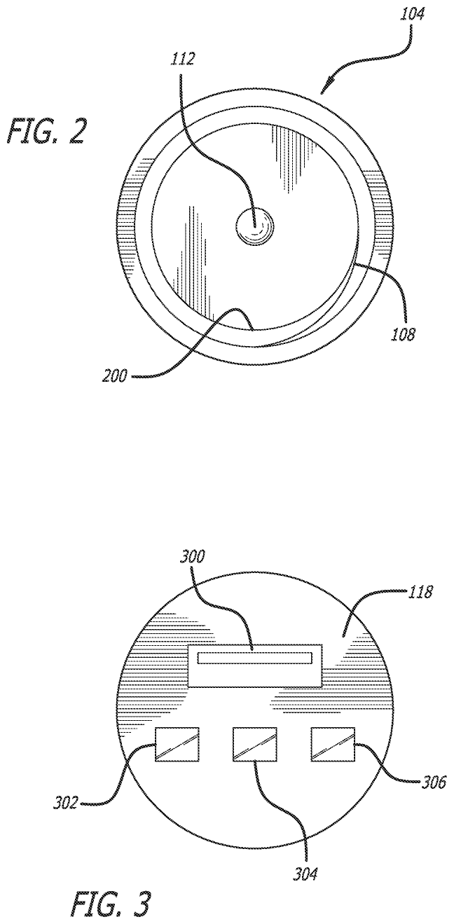

Reference is made to FIG. 1, which depicts a disinfection device 100 configured to disinfect a hub of a medical device using ultraviolet light. The disinfection device 100 includes a housing 102. In one aspect, the housing 102 may include an elongate cylindrical shape having a distal end 104 and a proximal end 106. The distal end 104 may include an opening or recess 110 configured to receive a portion of a hub of a medical device. A connection mechanism 108 may be located within or around the opening or recess 110. The connection mechanism 108 may be configured to removably attach or connect to a hub of a medical device. In some aspects, the hub may include a needleless connector. In some aspects, the hub may comprise a male or female Luer connector. In other instances, the hub may comprise a PRN connector. An example of a hub is the Q-SYTE™ Luer access port from Becton, Dickinson, and Company. Further, in some instances a hub may comprise a port or valve of a section of intravenous tubing or a connector thereof. One having skill in the art will appreciate that the systems and methods of the present invention may be adapted for use with various other types of connectors, hubs and other devices for which disinfection is desirable.

In some aspects, the housing 102 may be formed of an easily cleanable material such that the disinfection device 100 may be cleaned between uses. In some aspects, the housing 102 may be formed of a germ free or microbe resistant material. In some aspects, the disinfection device 100 may be configured to self-disinfect between uses.

The connection mechanism 108 allows for a hub in need of disinfection to be connected to the disinfection device 100. The connection mechanism 108 may include one or more threaded connection, screw thread, channel, ridge, friction fit, magnet, and ferromagnetic material. For example, the connection mechanism 108 may include a Luer type connector. The connection mechanism 108 may be configured to contact an end, tip, outer surface, or inner surface of a hub of a medical device. In some aspects the connection mechanism may allow for encapsulation of a hub of a medical device.

In some aspects, the connection mechanism 108 may be located around the perimeter of inner surface of the opening or recess 110. In other aspects, the connection mechanism 108 may be positioned on an outer surface of the distal end 104 of the disinfection device 100 such that the distal end 104 is inserted into a hub during disinfection. In some aspects, the connection mechanism 108 may completely surround the opening or recess 100. In other aspects, the connection mechanism 108 may only be located on a portion of the opening or recess 100.

The disinfection device may include an ultraviolet source 112 located near the distal end 104 in order to impart ultraviolet radiation into a hub requiring disinfection in order to render any microorganisms within or around the hub innocuous. In some aspects, ultraviolet source 112 comprises a biocidal lamp. In some aspects, the ultraviolet source 112 may include a UV light emitting diode having a peak of wavelength within the range of 100-400 nm. Additionally, some specific embodiments may include a germicidal ultraviolet light (UV-C) having a wavelength range of substantially 225-264 nm. As used herein, the term “ultraviolet source” is used to denote a lamp, a light-emitting diode (LED), a laser, or another similar technology that is capable of emitting wavelengths in the range of 290 nm-100 nm, and/or which are capable of killing pathogens present.

In some aspects, the ultraviolet source 112 may be housed behind a lens or window. In some aspects, an interior surface of the opening or recess 110 may include a reflective material configured to reflect and aim the ultraviolet light at a hub connected to the connection mechanism 108. In some embodiments, interior surface 200 of the distal end 104 of the housing 102 may include a UV-C reflective material, or other materials with high UV-C reflective properties. UV-C reflective material may be provided to retain and reflect the UV-C radiation emitted by ultraviolet source 112 within the housing 102 and guide it towards an attached hub needing disinfection. Further, use of UV-C reflective material provides greater intensity and exposure of UV-C radiation to an attached hub resulting in efficient irradiation of microbes during the disinfection regimen.

In some aspects, the disinfection device 100 may include memory and be preprogrammed with exposure time and power settings to ensure disinfection for various hubs. The disinfection device 100 may include a processor 116 configured to control the ultraviolet source 112 at a desired power for a desired duration of time depending on what type of hub is requiring cleaning. In some aspects, the disinfection device 100 may recognize the type of hub requiring cleaning upon connection to the hub and initiate a preset disinfection routine that applies the proper ultraviolet exposure to ensure disinfection.

In one aspect, the disinfection device 100 may include a sensor, camera, RFID reader, barcode scanner, transmitter, or transceiver to aid in the identification of an attached hub in need of disinfection. For example, various hubs may be provided with a bar code, QR code, RFID tag, or other descriptive information that may be received by the disinfection device 100 upon connection of the hub to the device 100. Once identified, the disinfection device 100 may perform the proper preset disinfection routine.

The proximal end 106 of the disinfection device 100 may include an end wall 118 as shown in FIG. 3. The end wall may include a port 300 for charging a rechargeable power supply 114 located within the housing 102 of the disinfection device 100. Additionally, information may be uploaded or downloaded from the disinfection device via port 300. In some aspects, the rechargeable power supply 114 may include a battery or capacitor. The port 300 may be configured to receive a power supply cable 120. In some aspects, the port 300 may be configured as a USB port that allows connection of the device to an external device, for example a CPU. In some aspects, the rechargeable power supply 114 may be configured to wirelessly recharge, for example, by inductance charging.

The disinfection device 100 may be configured to power on manually or automatically. In one aspect, the disinfection device 100 may power up and begin delivering ultraviolet radiation to an attached hub by actuating an on/off button or switch 122. In another aspect, the disinfection device 100 may be configured to automatically power up and begin a disinfection regimen upon attachment with a hub of a medical device. In some embodiments, a switch or sensor located within the opening or recess 110 may be triggered when the disinfection device 100 is attached to the hub resulting in initiation of the disinfection regimen.

As illustrated in FIG. 3, a plurality of indicators 302, 304, 306 may be located on the end wall 118. The plurality of indicators 302, 304, 306 may provide an indication of the operating status of the disinfection device 100. For example, indicator 302 may be configured to illuminate when the device 100 has been powered on, indicator 304 may signal that the ultraviolet source 112 is operating, and indicator 306 may flash when disinfection of an attached hub is complete. While three indicators 302, 304, 306 are shown, it is understood that more or less may be provided. The plurality of indicators 302, 304, 306 may be visible light spectrum light emitting diodes having at least one color. In some aspects, each of the plurality of indicators 302, 304, 306 may be configured to have more than one color. In some aspects, at least one of the plurality of indicators 302, 304, 306 may indicate a level of disinfection of an attached hub. In some instances, at least one of the plurality of indicators 302, 304, 306 may blink to indicate a stage or level of disinfection for an attached hub. In some aspects, the plurality of indicators 302, 304, 306 may comprise two or more colors, wherein a color indicates a stage or level of disinfection for a medical device hub. For example, in some embodiments a red light indicates incomplete or unsatisfactory disinfection while a yellow light may indicate an intermediary or active process of disinfection for an attached hub. Further still, a green light may indicate a satisfactory or complete disinfection.

The plurality of indicators 302, 304, 306 may further be programmed to blink or otherwise demonstrate a lighted pattern to further communicate a status of the disinfection device 100. For example, the plurality of indicators 302, 304, 306 may be programmed to demonstrate a lighted pattern to indicate a low battery. In some aspects, the plurality of indicators 302, 304, 306 may further be programmed to demonstrate and error or mechanical malfunction. In some instances, the plurality of indicators 302, 304, 306 may be programmed to indicate that disinfection device 100 is ready to receive a hub of a medical device. In place of the plurality of indicators 302, 304, 306, information to a user may be provided by a display screen (not shown), such as a LCD screen, that displays operation times, disinfection status, battery levels, or other such status notifications.

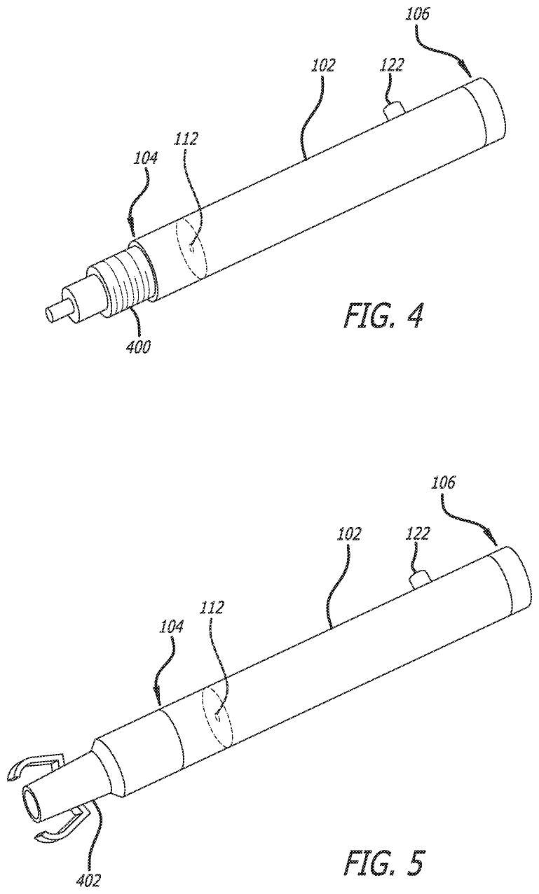

FIG. 4 illustrates the disinfection device 100 being coupled to a male Luer connector 400. Male Luer connector 400 is attached to the disinfection device 100 so as to allow disinfection of internal surfaces of the male Luer connector 400.

FIG. 5 illustrates the disinfection device 100 being coupled to a female Luer connector 402. Feale Luer connector 400 is attached to the disinfection device 100 so as to allow disinfection of internal surfaces of the female Luer connector 400.

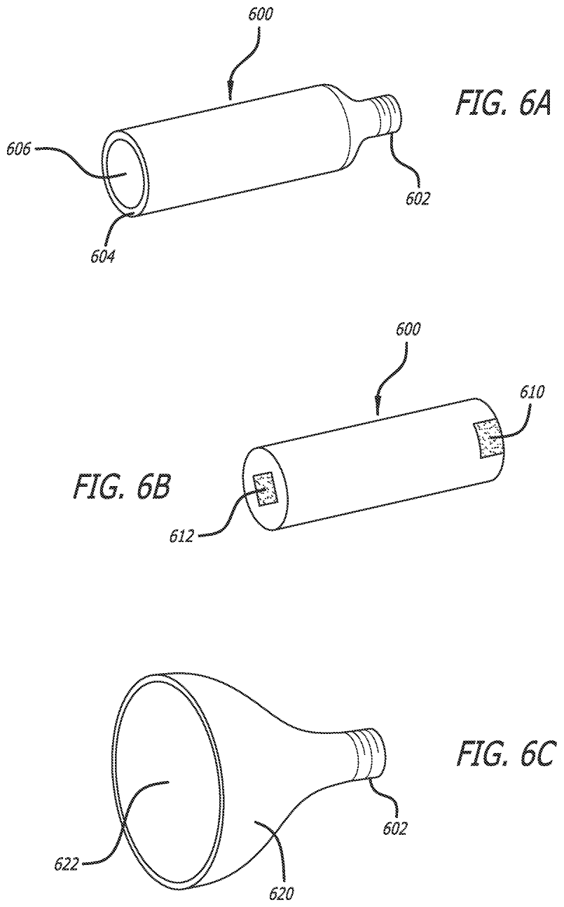

In some aspects, a sterile adapter may be configured to connect the distal end 104 of the disinfection device 100 to a hub of a medical device. FIG. 6A illustrates an adapter 600 having a threaded proximal end 602 configured to couple to the connection mechanism 108 of the disinfection device 100. The distal end 604 of the adapter 600 may include an opening or recess 606 configured to receive a portion of a hub of a medical device that requires disinfection. The distal end 604 may also include a screw thread or other connection mechanism to removably connect to a hub.

FIG. 6B illustrates an adapter 600 magnetically coupled to the disinfection device 100. In one aspect, the adapter 600 includes one or more magnet 610 on a proximal end to couple with a ferromagnetic material associated with the distal end 104 of the disinfection device 100. Alternatively, the adapter 600 may include a ferromagnetic material that couples with one or more magnet located at the distal end 104 of the disinfection device. Adapter 600 may also include one or more magnet or ferromagnetic material 612 positioned at a distal end of the adapter to couple with a hub of a medical device.

FIG. 6C illustrates an adapter 600 configured to encapsulate or surround a hub of a medical device in need of disinfection. The adapter 600 may include a connection mechanism in the form of a threaded proximal end 602 configured to couple with the connection mechanism 108 at the distal end of the disinfection device 100. In other aspects, the connection mechanism may take the form of a ridge, recess, magnet, or ferromagnetic material. A distal portion may include a flange 620 having an opening 622 into which at least a portion of a hub for a medical device can be positioned.

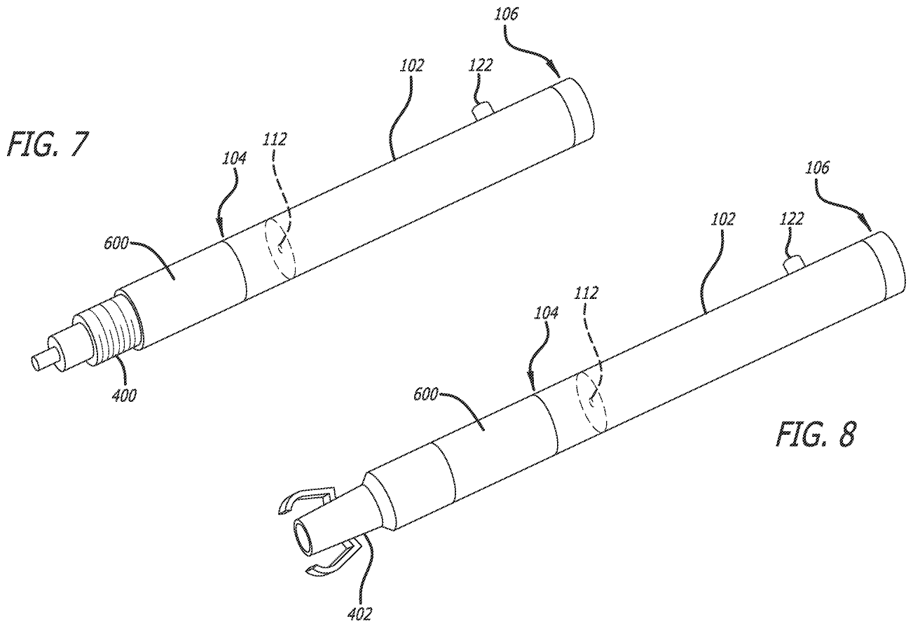

FIG. 7 illustrates a disinfection device 100 having an adapter 600 being coupled to a male Luer connector 400. The adapter 600 is configured to transfer ultraviolet light from the ultraviolet source 112 within housing 102 to the male Luer connector 400 for disinfection. The adapter 600 may include a UV-C reflective material or fibers to transmit the ultraviolet light from the disinfection device 100 to the male Luer connector 400.

FIG. 8 illustrates a disinfection device 100 having an adapter 600 being coupled to a female Luer connector 402. The adapter 600 is configured to transfer ultraviolet light from the ultraviolet source 112 within housing 102 to the female Luer connector 402 for disinfection. The adapter 600 may include a UV-C reflective material or fibers to transmit the ultraviolet light from the disinfection device 100 to the male Luer connector 400.

Some embodiments of the present invention further comprise a device or component of a device that includes a material, a coating, or a tag containing a material or coating that is configured to change color in response to prolonged exposure to air and/or a liquid. A color-changing material may be useful in communicating to a user a length of time for which the shield and/or needleless connector has been exposed to an unsterile environment. A color-changing material may also be useful in communicating to a user that the device or component has been previously used. In some instances, a device or component of the present invention is packaged in airtight packaging, thereby preserving an initial color of the color-changing material. Upon opening the airtight packaging, the color-changing material is exposed to air thereby changing the color of the device or component. This feature may prevent a device or component from being reused. This feature may also prevent an unsterile device or component from being used.

It should therefore be understood that these and other variations of the principles described herein are contemplated and that the cross-sectional profiles of the multi-lumen catheter tubes disclosed herein can vary as appreciated by one skilled in the art.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A disinfection device for disinfecting a hub of a medical device, comprising:
a housing having a first end and a second end;
a power source;
an ultraviolet light source; and
a connection mechanism located at the first end of the housing, wherein connection of the hub of the medical device to the disinfection device initiates a disinfection process configured to disinfect the hub.

2. The disinfection device of claim 1, wherein the ultraviolet light source is a light emitting diode with a peak of wavelength within the range of 100-400 nanometers (nm).

3. The disinfection device of claim 1, wherein the connection mechanism includes one or more of a screw thread, a channel, ridge, a friction fit connector, a magnet, or a ferromagnetic material.

4. The disinfection device of claim 1, further comprising a port located on the second end, wherein the one or more port is configured for coupling to an external device and receiving a charge or power supply from the external device.

5. The disinfection device of claim 1, wherein the power source is a battery, a capacitor, or an induction coil.

6. The disinfection device of claim 1, wherein the connection mechanism is configured for attaching the disinfection device to the hub of the medical device.

7. The disinfection device of claim 1, further comprising:
at least one indicator being one or light emitting diodes (LEDs) configured to indicate either a power level of the power source or a status of the disinfection process.

8. The disinfection device of claim 1, wherein connection of a hub of a medical device with the connection mechanism initiates disinfection of the hub.

9. The disinfection device of claim 1, further comprising a transceiver configured to obtain an identifier from the medical device, wherein the identifier indicates a preprogrammed disinfection regimen to be performed by the disinfection device.

10. The disinfection device of claim 1, wherein the transceiver includes either an RFID or BLUETOOTH® enabled transmitter or transceiver.

11. A method of disinfecting a hub of a medical device, the method comprising:
providing a disinfection device for disinfecting a hub of a medical device, comprising:
a housing having a first end and a second end,
a power source,
an ultraviolet light source, and
a connection mechanism located at the first end of the housing, wherein connection of the hub of the medical device to the disinfection device initiates a disinfection process configured to disinfect the hub;
establishing a coupling between the medical device and the disinfecting device; and
enabling the disinfection device to perform the disinfection process thereby disinfecting the hub.

12. The method of claim 11, wherein the ultraviolet light source is a light emitting diode with a peak of wavelength within the range of 100-400 nanometers (nm).

13. The method of claim 11, wherein the connection mechanism includes one or more of a screw thread, a channel, ridge, a friction fit connector, a magnet, or a ferromagnetic material.

14. The method of claim 11, wherein the disinfecting device further comprises a port located on the second end, wherein the one or more port is configured for coupling to an external device and receiving a charge or power supply from the external device.

15. The method of claim 11, wherein the power source is a battery, a capacitor, or an induction coil.

16. The method of claim 11, wherein the connection mechanism is configured for attaching the disinfection device to the hub of the medical device.

17. The method of claim 11, wherein the disinfecting device further comprises at least one indicator being one or light emitting diodes (LEDs) configured to indicate either a power level of the power source or a status of the disinfection process.

18. The method of claim 11, wherein connection of a hub of a medical device with the connection mechanism initiates disinfection of the hub.

19. The method of claim 11, wherein the disinfecting device further comprises a transceiver configured to obtain an identifier from the medical device, wherein the identifier indicates a preprogrammed disinfection regimen to be performed by the disinfection device.

20. The method of claim 11, wherein the transceiver includes either an RFID or BLUETOOTH® enabled transmitter or transceiver.
